(19) United States
(12) Patent Application Publication
(10) Pub. No.: US 2013/0218106 A1
(43) Pub. Date: Aug. 22, 2013

(54) Automated Method of Pooling Elimination with a Biological Fluid Collection System

(71) Applicants: Anthony Coston, Dunstable, MA (US); Paul Ciccone, Social Circle, GA (US)

(72) Inventors: Anthony Coston, Dunstable, MA (US); Paul Ciccone, Social Circle, GA (US)

(73) Assignee: C. R. BARD, INC., Murray Hill, NJ (US)

(21) Appl. No.: 13/703,621

(22) PCT Filed: Jul. 29, 2011
(86) PCT No.: PCT/US11/45956

§ 371 Date: May 3, 2013

Related U.S. Application Data
(60) Provisional application No. 61/369,494, filed on Jul. 30, 2010.

(51) Int. Cl. A61M 1/00

(52) U.S. Cl. CPC A61M 1/00
USPC 604/317

(57) ABSTRACT

A drainage and/or collection system for biological fluids includes at least one conduit for transporting a biological fluid from a catheter to a collector device and a gas pressure source configured to feed a gas into the at least one conduit between the catheter and the collection device. The gas causes the biological fluid arranged in the at least one conduit to drain into the collection device. A method includes inserting a catheter, draining a fluid into a collector device via a conduit, and introducing gas into the conduit so as to force fluid remaining in the conduit into the collection device.

### CROSS REFERENCE TO RELATED APPLICATIONS

This application claims benefit of priority to U.S. Provisional Application No. 61/369,494, filed Jul. 30, 2010, and entitled “Automated Method of Pooling Elimination with a Biological Fluid Collection System,” the contents of which are incorporated herein by reference.

### STATEMENT CONCERNING GOVERNMENT INTEREST

Not applicable.

### BACKGROUND OF THE INVENTION

Catheterization is a sterile process of draining urine from the bladder. Typically, a catheter is inserted into a bladder so that fluid can pass out through the catheter, into a conduit and then into a collection vessel. The amount of urine in the collection vessel is then measured.

With known systems, a significant amount of urine can remain or pool in the conduit and does not easily pass into the collection vessel. As such, it is difficult to determine accurately how much urine actually exited from the bladder. Urine output readings can thus not be accurately determined this way.

While it is possible to manipulate or move (or “milk”) the conduit so that some urine trapped in the conduit can be forced or flushed via gravity into the collection vessel, this method is generally limited because it can be difficult to remove all or most of the urine in the conduit due to limited venting, and because some urine will necessarily adhere to the inner wall of the conduit due to, e.g., surface tension. Also, this pooling of fluid within the conduit typically forces a clinician to intervene in order to force fluid into the collection vessel. This additional effort required by the physician negatively impacts clinician efficiency.

What is needed is a more reliable, consistent and easier way to accurately measure collected biological fluid such as urine. What is needed is a system and method to move pooled fluid into the collection vessel using a gas in order to more accurately determine a quantity or volume of removed fluid. What is needed is a system and method which can more reliably and easily be used to accurately collect a fluid such as urine from a user. What is also needed is a system that reduces or eliminates the need for user intervention.

### SUMMARY OF THE INVENTION

According to one non-limiting embodiment of the invention, there is provided a drainage system for biological fluids which comprises a control device for supplying continuous or intermittent gas flow, e.g., a steady stream or pulses of air, to at least one conduit structured for transporting a biological fluid from a catheter to a collector device in order to eliminate pooling of the biological fluid within the at least one conduit. The gas forces the biological fluid pooling in the at least one conduit to drain into the collection device.

According to one non-limiting embodiment of the invention, there is provided a drainage and/or collection system for biological fluids which comprises at least one conduit for transporting a biological fluid from a catheter to a collector device and a gas pressure source configured to feed a gas into the at least one conduit between the catheter and the collection device. The gas causes the biological fluid arranged in the at least one conduit to drain into the collection device.

In embodiments, a pressure of the gas exiting the gas pressure source is at least greater than atmospheric pressure and having the form of a single pressure pulse, greater than atmospheric pressure and having the form of a gas flow which occurs for a predetermined amount of time, greater than atmospheric pressure and having the form of a gas flow which occurs for between about 1 second and about 10 seconds, greater than atmospheric pressure and having the form of a single pressure pulse, and sufficiently high so as to cause substantially all fluid in the at least one conduit to drain into the collection device.

Embodiments of the invention are directed to a drainage or collection system for biological fluids. The system includes at least one conduit for transporting a biological fluid from a catheter to a collection device, and an automated device programmable to automatically supply at least one gas pulse through the at least one conduit and into the collection device.

According to embodiments, the automated device can include a programmable microprocessor coupled to control a gas source. Further, the gas source may include a vacuum pump. The automated device can also include a pressure transducer structured and arranged to monitor the gas pressure of the at least one gas pulse.

In accordance with embodiments of the invention, the automated device may include a user interface to program at least one of gas pressure magnitude, gas pulse duration, and period between pulses.

According to further embodiments, a valve may be located between the catheter and the container to prevent the at least one gas pulse from flowing toward the catheter.

According to other embodiment of the instant invention, the automated device may include a gas pulse control or regulation device comprising a pressure transducer and a microprocessor.

The system can also include a transducer positionable at least partially beneath the collector device. Moreover, an output of the transducer can be input to the automated device.

In accordance with still other embodiments of the present invention, the collector device may include a filter and a closable filter cover. The collector device can also include a drain tube, extending from a bottom of the collection device, having an end insertable into a fluid reservoir. The collector device may also include a high level sensor coupled to the automated device. Alternatively or additionally, the collector device can also include a low level sensor coupled to the automated device.

Moreover, the automated device may include a signal conditioning circuit structured to receive at least one of bladder pressure and bladder temperature as an input. The signal conditioning circuit may be coupled to a gas source structured and arranged to generate the at least one gas pulse.

The invention is directed to a method for draining or collecting biological fluids. The method can include guiding biological fluid through at least one conduit from a catheter to a collection device, and automatically supplying at least one gas pulse through the at least one conduit and into the collection device.

According to embodiments of the instant invention, the at least one gas pulse can force biological fluids pooling in the at least one conduit into the collection device. Additionally or alternatively, the at least one gas pulse can force biological fluids in the collector device out of the collection device.

In accordance with other embodiments, the method can also include programming a microprocessor to control a gas source to generate the at least one gas pulse.

Embodiments of the method can also include controlling or regulating a pressure magnitude of the at least one gas pulse.

According to still further embodiments, the method may include programming at least one of gas pressure magnitude, gas pulse duration, and period between pulses.

In accordance with further embodiments, the method can include measuring a volume of the fluid in the collection device. The method can also include forwarding the measured weight of the collector device an output of the transducer is input to the microprocessor.

In accordance with further embodiments, wherein the volume of fluid is measured with an ultrasonic device, and the method further comprises forwarding emitted and received pulses to the microprocessor; determining a time of flight between the emitted and received pulses; and determining the fluid volume from the time of flight. According to other embodiments of the instant invention, the method can include closing a closable filter cover over a filter located in the collection device.

According to further embodiments, the method can include monitoring a high level sensor of the collection device, and issuing an alert when the biological fluids reach the high level sensor.

In accordance with still yet other embodiments of the present invention, the method can include inputting at least one of bladder pressure and bladder temperature into a signal conditioning circuit coupled to the gas source.

In embodiments, the catheter is a Foley catheter and the biological fluid is urine.

In embodiments, the system and method is utilized on a collection system of the type disclosed in US 2007/0010797 to NISHTALA et al., the disclosure of this document is expressly incorporated by reference herein in its entirety.

In embodiments, the system and method is utilized on a collection system of the type disclosed in U.S. Pat. No. 3,961,529 to HANIFL, the disclosure of this document is expressly incorporated by reference herein in its entirety.

In embodiments, the system and method utilizes a sampling coupling device of the type disclosed in U.S. Pat. No. 4,423,741 to LEVY, the disclosure of this document is expressly incorporated by reference herein in its entirety.

In embodiments, the system and method utilizes on a communication control system of the type disclosed in U.S. Pat. No. 4,819,653 to MARKS, the disclosure of this document is expressly incorporated by reference herein in its entirety.

In embodiments, the system and method utilizes a catheter of the type disclosed in U.S. Pat. No. 4,227,533 to GODFREY, the disclosure of this document is expressly incorporated by reference herein in its entirety.

In embodiments, the system and method utilizes one or more one-way valves of the type disclosed in U.S. Pat. No. 6,240,960 to FILLMORE and U.S. Pat. No. 6,481,462 to FILLMORE et al., the disclosures of this document are each expressly incorporated by reference herein in their entireties.

### BRIEF DESCRIPTION OF DRAWINGS OF THE EXEMPLARY EMBODIMENTS

FIG. 1 shows a system for draining and flushing a biological fluid in accordance with a non-limiting embodiment of the invention;

FIG. 2 shows in more detail the automated device depicted in FIG. 1;

FIG. 3 shows another non-limiting embodiment of the invention;

FIG. 4 shows a further non-limiting embodiment of the invention;

FIG. 5 shows non-limiting embodiments of flow diagram depicting various processes in accordance with the invention;

FIG. 6 shows further non-limiting embodiments of flow diagrams depicting various further processes in accordance with the invention

### DETAILED DESCRIPTION OF THE EXEMPLARY EMBODIMENTS

The following description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will enable one skilled in the art to make and use the invention, and describes several embodiments, adaptations, variations, alternatives and uses of the invention, including what is presently believed to be the best mode of carrying out the invention.

As used herein, the reference terms “proximal” and “distal” (proximal being closer than distal) refer to proximity with respect to a health care professional catheterizing a patient. For example, the region or section of the catheter apparatus that is closest to the health care professional during catheterization is referred to herein as “proximal,” while a region or section of the catheter apparatus closest to the patient's bladder is referred to as “distal.” In the case of a self-catheterizing patient, proximal refers to a point external to the patient's body, and distal refers to a point within the patient's body (i.e., the bladder).

Embodiments of the invention can be utilized in conjunction with known catheter draining systems. In an exemplary embodiment, embodiments of the invention can be used with a catheter draining system for draining urine from a patient's bladder through an inserted urinary catheter as described in commonly owned U.S. Provisional Application No. 61/289,869 filed Dec. 23, 2009, the disclosure of which is expressly incorporated by reference herein in its entirety. However, it is noted that the instant invention is not limited to urinary catheter applications, such that other draining systems can be alternatively utilized without departing from the spirit and scope of the invention.

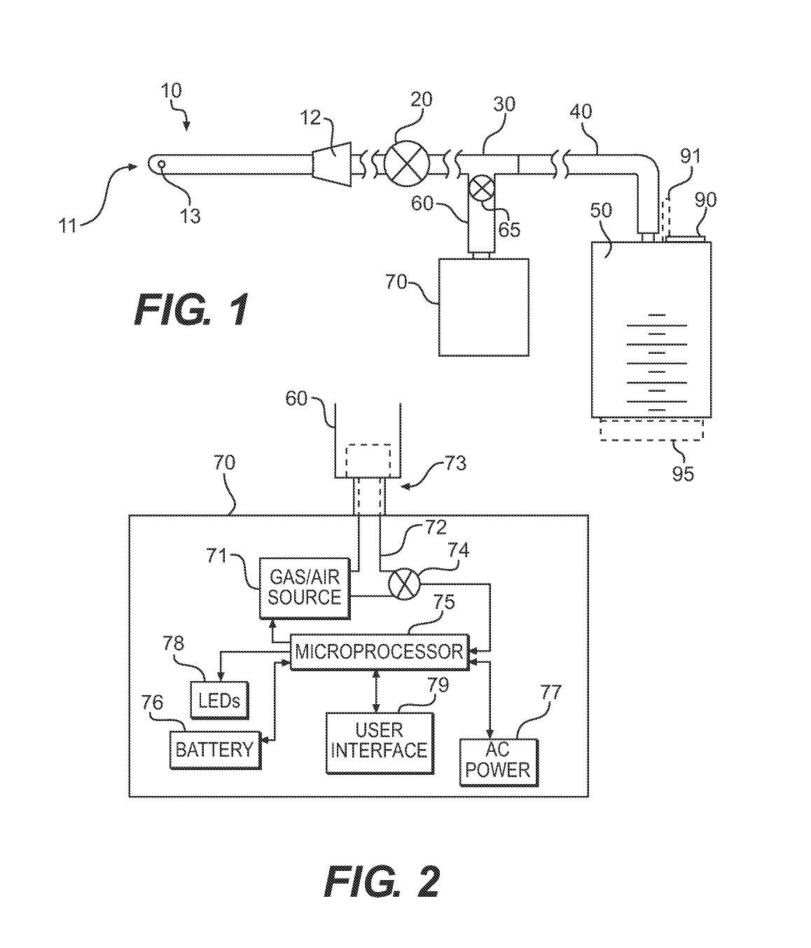

FIG. 1 shows a non-limiting embodiment of a catheter draining system 1 in accordance with the present invention. The system 1 utilizes a catheter 10 having a distal end 11 for insertion into, e.g., a bladder, and a proximal end 12 which includes an exit opening allowing a fluid, e.g., urine in a bladder, to pass out of the catheter 10. One or more drainage openings 13 are arranged on the distal end 11 allow fluid to pass into the catheter 10. Any type of catheter, whether known or otherwise, can be utilized provided it functions with the system components of the type described herein.

The system 1 also utilizes a device 20 that allows fluid to pass from the catheter 10 to a collector device 50 that collects the fluid removed with the catheter 10. Device 20 is structured to prevent fluid from passing back into the catheter 10. By way of a non-limiting example, the device 20 is a one-way valve. In embodiments, the device 20 can be hydrophobic filter. In further embodiments, the device 20 can be a one-way valve of the type disclosed in U.S. Pat. No. 6,240,960 to FILLMORE and/or U.S. Pat. No. 6,481,462 to FILLMORE et al., the disclosures of which are each expressly incorporated by reference herein in their entireties. In other embodiments, the device 20 can have a configuration similar to the sampling coupling device disclosed in U.S. Pat. No. 4,423,741 to LEVY, the disclosure of which is expressly incorporated by reference herein in its entirety.

The system 1 also utilizes a connection device 30, e.g., a “T” fitting, which has one end coupled to the device 20, another end coupled to a conduit 40 which is in fluid communication with the collector device 50, and still another end coupled to a conduit 60 which is in fluid communication with an automatic system 70. Automatic system 70 can include a gas/air pressure supply and a control device for controlling the gas/air pressure supply. The conduit 40 (as well as the conduit sections connecting device 20 to T fitting 30) can be any type of tubing typically utilized in conventional biological fluid draining systems, e.g., ¼″ to ⅜″ tubing. Further, conduit 60, which supplies gas/air pressure to through conduit 40, can be e.g., ⅛″ to ¼″ tubing.

The collector device 50 can be any type of container typically utilized in fluid collection devices. In embodiments, the collector device 50 has indicia that allow a user to accurately measure the amount of fluid inside. According to various embodiments, collector device is mounted on the bed, e.g., hooked onto a bedside rail, on the floor, or resting on the patient. In embodiments, one end of the conduit 40 is coupled to a top end portion of the collector device 50 so that fluid entering the collector device 50 will settle at the lowest point and provide for an accurate measurement of the quantity or volume of fluid in the collector device 50.

In operation, a fluid from the patient's body can be carried from, e.g., the bladder, to collector device 50 through catheter 10, device 20, and conduit 40. However, because these fluids generally tend to pool in conduit 40, an accurate reading of the amount of fluid leaving the patient's body cannot be made without the caregiver or other personnel manipulating conduit 40 to urge the pooled fluid into collector device 50. However, this manipulation can sometimes cause an inadvertent pulling on the catheter that can result in discomfort to the patient.

To avoid this need to manipulate the pooled fluid in conduit 40, gas or air pressure in the form of, e.g., continuous or intermittent pulses, can be generated and controlled by automatic device 70 up through connection device 30. As device 20 is structured to allow unidirectional flow from catheter 10 to collector device 50, the gas/air supplied by automatic device 70 will not pass back though catheter 10 to cause the patient any discomfort, but is guided through conduit 40 to push the pooled fluid into collector device 50 so that an accurate determination of the fluid can be made. A valve 65, e.g., a one-way valve in the air lumen to prevent backflow of fluid into the air line and ultimately into the electronic pump, may also be provided in connection device 30 to allow fluid to freely flow from device 20 toward conduit 40 but prevents any flow from device 20 into conduit 60. Valve 65 may further allow gas or air to flow from automatic device 70 through connector 30 and into conduit 40. Moreover, device 20 and valve 65 can be combined into a single device to allow the one way flow of fluid into conduit 40 and the one way flow of air into conduit 40. Collector device 50, which can be a rigid or semi-rigid structure, can be provided with an air outlet 90 that allows the gas or air passing through conduit 40 and into collector device 50 to escape, while the fluid remains in collector device 50. Air outlet 90 can also include a hydrophobic filter to allow the gas or air to escape from collector device 50. Further, a transducer 95, e.g., an ultrasonic transducer such as that used in the CRITICORE® Monitoring System by the assignee of the present invention C. R. Bard, can optionally be positioned under collector device 50 to monitor fluid volume changes within collector device 50. As a rigid or semi-rigid structure, collector device 50 generally maintains its a constant internal volume during the collection/monitoring process. To monitor fluid volume changes, transducer 95 can send ultrasonic pulses through the bottom of collector device 50 and into the fluid contained within collector device 50. When the ultrasonic pulse hits the fluid/air interface within collector device 50, the pulse bounces back and is captured by transducer 95. From a determination of the time of flight (TOF) between the outgoing and returning pulses, the system can determine the volume of collected fluid. In the exemplary embodiment, time of flight of the ultrasonic pulse within collector device 50 is determined by automatic system 70 from the pulse data sent from transducer 95. Further, as the dimensions of collection container are generally fixed to maintain a constant internal volume, the time of flight data can be correlated to a predefined fluid volume for an accurate determination of the amount of fluid within collection container 50. By way of non-limiting example, as an area of the base of the collector device 50 can be predetermined, the time of flight determines the height of the fluid, such that the volume can be easily calculated. Since the ultrasonic pulses occur multiples times per second, transducer 95 coupled with the automatic system 70 can be used to indicate and/or monitor changes in volume, which can be used as an indication that fluid is flowing into collector device 50.

A non-limiting exemplary embodiment of automatic system 70 is illustrated in FIG. 2. As shown, automatic system can be connected to conduit 60 via a connector 73, e.g., a Luer fitting or connector, so that a channel 72 connects conduit 60 a gas or air source 71, e.g., a pressure vacuum or a pump, e.g., rotary vane pump (G 01-K-LC) manufactured by Thomas Co. In this manner, gas or air from source 71 can be supplied through channel 72 and conduit 60 and guided through conduit 40 into collector device 50. As a result of the gas or air traveling through conduit 40, any fluids pooling in conduit 40 will be forced out of conduit 40 and into collector device 50. A microprocessor 75 can be provided to control the gas or air output by gas or air source 71. In this regard, the gas or air can be a steady continuous stream of gas or air for a predetermined period of time or continuously in operation; can be a continuous stream of pulses of gas or air for a predetermined period of time or continuously in operation; and/or can be combinations thereof. Thus, automatic system 70 can provide a lightweight low cost system capable of producing the necessary gas or air pressure on a continuous or programmed intermittent basis.

The power source can take the form of a battery 76 and/or an ac adapter 77 which plugs the device into a wall outlet. Battery 76 can be e.g., a lithium ion or other rechargeable battery, and can be used as a main power supply or as a backup supply. Automatic system 70 can also include one or a number of LEDs to provide a visual indicator of the status of various processes, e.g., the device is on, the battery is low, ac power on, battery charging, etc.

Microprocessor 75 can be programmed through an interface 79, which can include at least one of, e.g., a touch screen, a keypad, a USB port, an Ethernet network connector, a wireless network connector, or other suitable interface to allow a user, caregiver and/or other personnel to set a gas or air stream strength and stream duration, and to turn the device on and off. Interface 79 can also include a display, e.g., an LCD display, to provide a visual indication or confirmation of the settings input by the user, caregiver and/or other personnel. The LCD display can also include an icon or other indicia to confirm that the status of various components of automatic system 70, e.g., battery charging/AC power on; battery power; battery charge, etc. However, in order to save the power required to continuously operate the LCD display, the LCD display can be put into a sleep mode to power down and conserve battery or electrical power. It is also contemplated that a pump algorithm can be hard-wired into the microprocessor so that a user cannot alter and/or access certain features to prevent harm through user error. Moreover, it is further contemplated to utilize a combination of these features, such that while certain features are unavailable for user modification or access, other features are provided for the user's input.

Opposite the output of gas or air supply 71, a pressure transducer 74 can be arranged to detect or monitor the magnitude of the gas or air pressure output by supply 71 through channel 72. Pressure transducer 74 can feedback a detected pressure magnitude to microprocessor 75 so that the gas or air supply can be controlled or regulated to the user or system defined pressure and for the user or system defined period.

As noted above, the gas or air pressure supply 71 will supply gas or air at a predetermined pressure for a period of time predetermined by the user, the caregiver, or other personnel. However, in further and/or alternative embodiments, the automatic system 70 can be programmed to operate until conduit is cleared. In a non-limiting embodiment, automatic system 70 can be programmed with, e.g., gas or air pressure magnitude (e.g., 1 psi) having a pulse duration (e.g., 5 sec.) and a delay time (e.g., 5 min.) between pooling eliminations. Because the pooling elimination in accordance with the embodiments increases the volume of the fluid within collector device 50 as the fluid drains into collector device 50. However, after the pooling is eliminated, the collector device will not increase in volume, i.e., the gas or air entering collector device 50 will escape through air outlet 90. As noted above, transducer 95 can be arranged to monitor the volume of collector device 50. Further, transducer 95 can communicate with automatic device 70 through a wired or wireless connection. In this manner, when gas or air is supplied to conduit 40 for eliminating pooling, the gas will be supplied until transducer 95 shows that the volume of collector device 50 is constant for, e.g., 5 seconds. Further, if there is no change in the volume within the collector device 50 discerned by transducer 95 at least 5 seconds after the gas or air pulse is triggered, microprocessor 75 will shut down gas or air supply 71 until the predetermined delay has elapsed.

Alternatively, or additionally, a load cell (not shown) can be arranged under collector device 50 to monitor the weight of collector device 50. As with the monitoring of fluid volume within collector device 50, pooling elimination in accordance with this embodiment can monitor increases in the monitored weight of the collector device 50 as an indication of fluid draining into collector device 50. Thus, after the pooling is eliminated, the weight of collector device 50 will not appreciably increase since the gas or air entering collector device 50 will escape through air outlet 90. As noted, load cell can be utilized as the lone monitoring device for collector device 50 by being arranged directly under collector device 50, or can be used in combination with transducer 95, e.g., such that transducer 95 is arranged directly on the bottom of collector device 50 and collector device 50 is position upon the load cell. The load cell can be arranged to monitor the weight of collector device 50 and can communicate with automatic device 70 through a wired or wireless connection. In this manner, when gas or air is supplied to conduit 40 for eliminating pooling, the gas will be supplied until transducer 95 shows that the weight collector device 50 is constant for, e.g., 5 seconds. Further, if there is no change in the volume within collector device 50 discerned by transducer 95 at least 5 seconds after the gas or air pulse is triggered, microprocessor 75 will shut down gas or air supply 71 until the predetermined delay has elapsed.

In other embodiments, automatic device 70 can also be utilized to assist in draining collector device 50. In this regard, collector device 50 should be emptied at least once a day, and generally multiple times daily. However, as this is generally a manual process that can be messy due to spills, splashes and contamination, embodiments of the invention provide a safer more efficient emptying process. By way of non-limiting example, the user, caregiver or other personnel can determine through observing the increasing fluid levels in container 50 that the container should be drained. In another non-limiting example, it is also contemplated that an indicator can be coupled to transducer 95 so that when the volume of fluid within and/or the weight of collector device 50 are indicative of a generally full container, an audio and/or visual indicator can be activated to alert the necessary personnel to empty container 50.

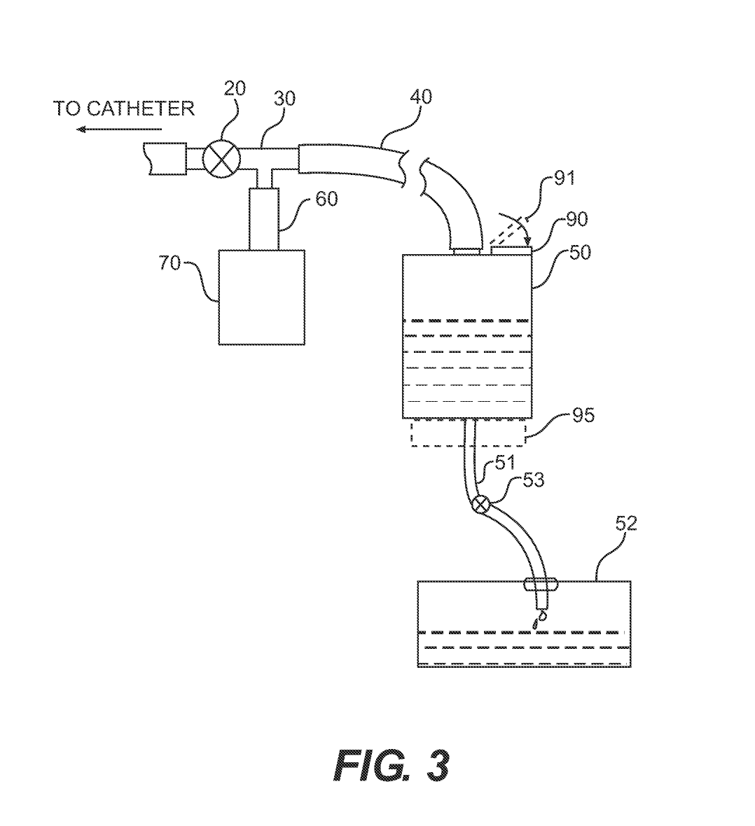

As illustrated in FIG. 3, a drain tube 51 extends from a bottom of collector device 50 into an external reservoir 52. External reservoir 52 can be transportable receptacle to collect the fluids drained from collector device 50, and is separable from collector device 50. A valve 53 can be located in drain tube 51 to that the user, caregiver, or other personnel can selectively open and close valve 53 in order to drain collector device 50. As the gas or air supplied into collector device 50 escapes through air outlet 90, collector device 50 (or air outlet 90) can include a filter cover 91 to prevent air from escaping from inside of collector device 50. Further, interface 79 can also include, e.g., an icon or other indicia selectable by the user, caregiver, or other personnel to instruct microprocessor 75 to activate gas or air source 71 in order to drain collection device in the manner described below.

In this manner, when filter cover 91 is in place over air outlet 90, external reservoir 52 can be placed below collector device 50 so that an end of drain tube 51 is inserted into an inlet port in external reservoir 52, and valve 53 can then be opened. Once valve 53 is opened, the fluid in collector device 50 will at best simply trickle out of drain tube 51. To assist in draining collector device 50, the user, caregiver or other personnel can press or otherwise select an icon or indicia associated with draining collector device 50, which can result in microprocessor 75 turning on gas or air source 71 at a predetermined collection device emptying pressure. The supplied gas or air will create a backpressure that travels through channel 72, conduit 60, and conduit 40 to not only force any pooled fluids into collector device 50, but also to force the fluid within collector device 50 out through drain tube 51 and into reservoir 52. In this regard, as the gas or air supplied into collector device 50 cannot escape through covered air outlet 90, the increasing gas or air pressure applied within collector device 50 will force the fluid in collector device 50 through drain tube 51 and into reservoir 52. Automatic system 70 can be operated manually, i.e., shut off (e.g., via the same icon or indicia; or another icon or indicia) after the user, caregiver or other personnel visually confirm that collector device 50 is empty. Alternatively, as the last of fluid leaves container 50, a pressure release will occur that can be detected by pressure transducer 74. Thus, once pressure transducer 74 detects the pressure release due to the last of the fluid exiting the drain tube, microprocessor 75 can shut down or deactivate gas or air source 71.

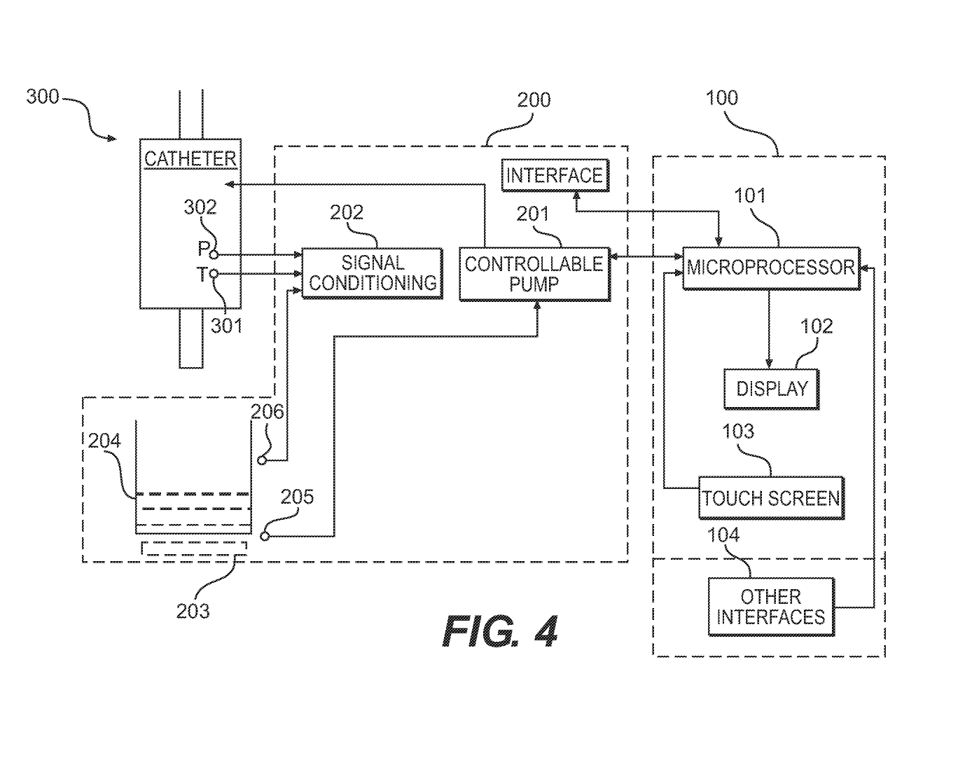

In further embodiments, FIG. 4 shows another non-limiting embodiment of an automated elimination of pooling in accordance with the invention. FIG. 4 generally shows a control device 100, a base station 200, and a catheter 300. Control device 100 can include a microprocessor 101, e.g., an AMD Geode LX 800, and at least one user interface, such as, e.g., display 102, such as an LCD display, and/or a touch screen 103. Control device 100 can also include a memory coupled to microprocessor 101 and the at least one user interfaces 102 and 103 to store software to facilitate a user's, caregiver's or other personnel's entry of data to configure desired operational parameters to be controlled by microprocessor 101. In this manner, the gas or air pressure for forcing pooling fluids out of the conduits and into the container can be set, as well as the duration of the applied pressure and/or the delay between the application of pressure to eliminate pooling in the conduit leading to the collection device.

Microprocessor 101 can be coupled to a controllable pump 201, e.g., an Atmel Xmega microcontroller, located within base station 200 remote through a connection, such as a serial connection. Base station 200 can be remote from control device 100 or control device 100 can be arranged on base station 200. Controllable pump 201 can be connected to receive data from a signal conditioning device 202 that receives data regarding bladder temperature 301 and bladder pressure 302 from catheter 300, as well as data from a transducer 203, e.g., a device for measuring fluid volume, such as that used in the CRITICORE® Monitoring System, arranged under collection device 204 to provide data regarding the volume of collection device 204. Further, it is understood that transducer 203 can also be, e.g., a load cell and/or a combination of a load cell and volume monitoring device. Collection device 204 can also include a low level indicator 205 coupled to controllable pump 201 and a high level indicator 206 coupled to signal conditioning device 202. In this regard, when the fluid level in collection device 204 reaches the level of high level indicator 205, signal conditioning device 202 can inform controllable pump 201 that it is time to empty collection device 204, and controllable pump 201 can inform microprocessor 101 to actuate an audio or visual alarm to indicate that collection device 204 should be emptied, e.g., in the manner described above. After emptying collector device 204, the low level indicator 205 can inform controllable pump 201 that collector device 204 is now empty and the pump should be turned off. Base station 200 can also include at least one interface, e.g., a USB port, an Ethernet network connector, a wireless network connector, or other suitable interface to allow a user, caregiver and/or other personnel to receive data from an interface other than on control device 100. By way of non-limiting example, the at least one interface on base station 200 can be used to connect to, e.g., hospital electronic medical records, so that the pump can be remotely set for operation.

The monitoring of bladder temperature 301 and bladder pressure 302 are generally well known in the art, and this information is utilized by the signal conditioning device 202 to additionally control controllable pump 201. The bladder temperature is determined by monitoring the output of a temperature sensor, e.g., a thermistor. In a particular embodiment, a thermistor, e.g., a YSI 400 series thermistor, can be used, which changes its resistance based on changes in temperature. The resistance value can be isolated, signal conditioned, and/or level shifted using typical methods of one normally skilled in the art. The pressure signal from the bladder can be transmitted through the catheter/tubing fluid column and may be detected by a pressure transducer. In a further embodiment, a GE NPC-100 pressure transducer can be advantageous. The pressure signal is isolated, signal conditioned, and or level shifted using typical methods of one normally skilled in the art.

Embodiments of the invention can also be directed to the method or process of eliminating pooling and/or emptying the collection device. Exemplary flow diagrams, which may represent a high-level block diagram of the embodiments, may be implemented and executed from the control device or from a server, in a client-server relationship, by computing devices in an ad hoc network, or they may run on a user workstation with operative information conveyed to the user workstation. Additionally, the invention can take the form of an entirely hardware embodiment, an entirely software embodiment or an embodiment containing both hardware and software elements. In an embodiment, the software elements include firmware, resident software, microcode, etc.

Furthermore, the invention can take the form of a computer program product accessible from a computer-usable or computer-readable medium providing program code for use by or in connection with a computer or any instruction execution system. The software and/or computer program product can be implemented in the environment comprising a microprocessor and a memory device. For the purposes of this description, a computer-usable or computer readable medium can be any apparatus that can contain, store, communicate, propagate, or transport the program for use by or in connection with the instruction execution system, apparatus, or device. The medium can be a tangible medium, such as an electronic, magnetic, optical, electromagnetic, infrared, or semiconductor system (or apparatus or device). Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

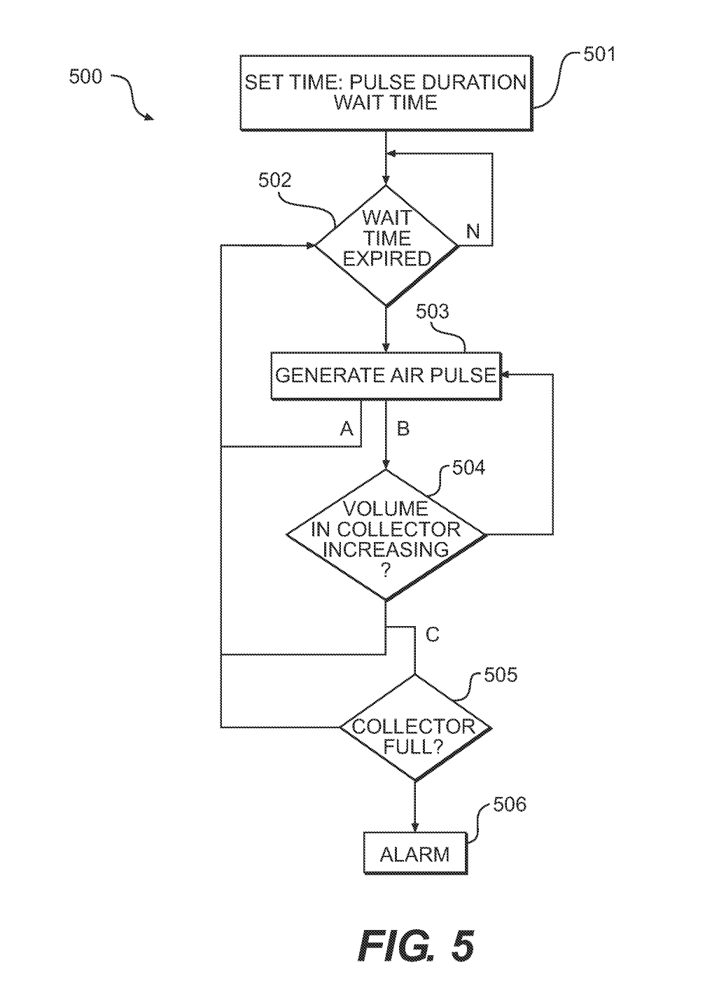

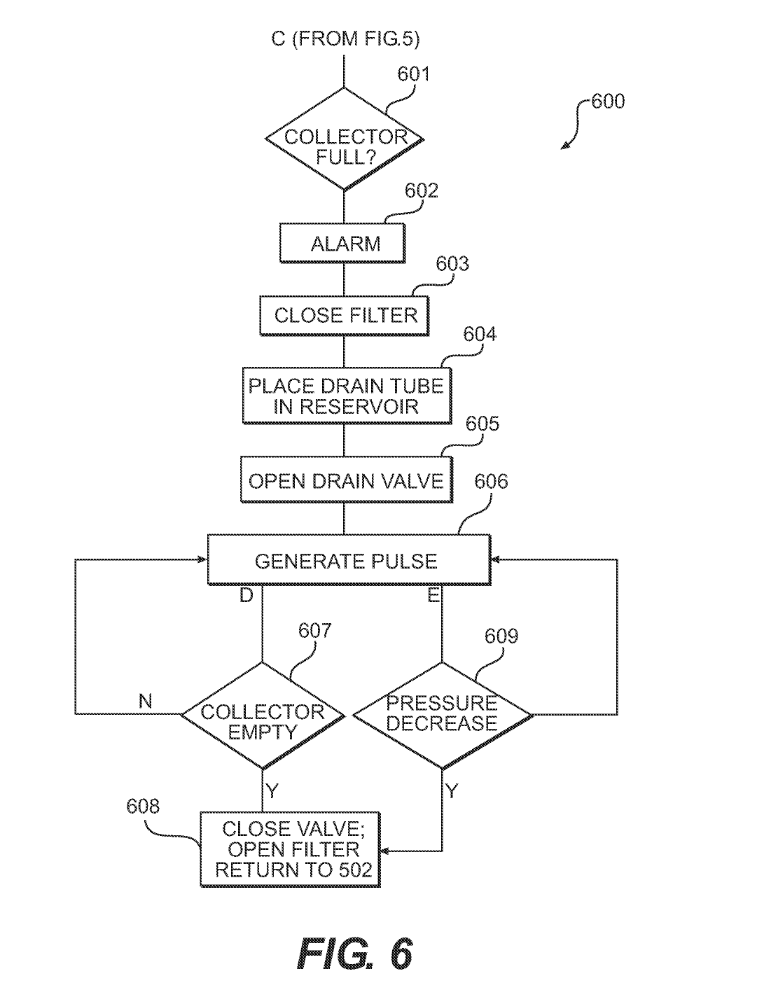

FIG. 5 shows a flow diagram 500 depicting steps of a non-limiting embodiment for eliminating pooling in the conduit leading to the collection device. At a step 501, the user, caregiver, or other personnel can set a timer on the interface for a control device for the gas or air pump. Setting the timer can include, e.g., setting a pulse duration; setting a wait period between pulses; setting a gas or air pressure magnitude for the pulse, e.g., 1 psi. At step 502, a determination is made whether the wait period has elapsed. If not, the system continues to wait. If the wait period has elapsed, a gas or air pulse is generated at step 503 at the set magnitude and duration into the conduit to be cleared of pooled fluid.

In a first optional embodiment, after the pulse is generated at step 503, the flow (as shown at point A) can return to step 502 to wait for the set delay to expire. In another optional embodiment, after the pulse is generated at step 503, the flow (as shown at point B) a determination can be made whether the volume {change to volume in FIG. 5} of the collection device is increasing at step 504. As noted above, as the pooling fluid is eliminated from the conduit, the fluid will increase the volume of the collection device. If the volume is still increasing after the pulse duration, the conduit is not completely empty, so the flow can return to step 503 to generate another pulse 504 to continue emptying the conduit. When the pulse duration ends and the volume is not increasing at step 504 the flow can return to step 502 to wait for the set delay to expire.

In a further optional embodiment, after the volume of the collection device is found not increasing at step 504, the flow (as shown a point C) can proceed to step 505 to determine whether the collection device is full. If not full, the flow can return to step 502 to wait from the set delay to expire. However, when the collection device is full, an audio and/or visual alarm can be turned on at step 506 to alert the user, caregiver, or other personnel that the collection device requires draining.

Another non-limiting exemplary embodiment of a flow diagram 600 is shown, which begins at point C in flow diagram 500. From point C, the flow diagram proceeds to step 601 to determine whether the collection device is full. The determination can be made from the volume of the collection device or from a level sensor. If not full, the flow can return to step 502 to wait from the set delay to expire. However, when the collection device is full, an audio and/or visual alarm can be turned on at step 602 to alert the user, caregiver, or other personnel that the collection device requires draining. At step 603, the filter cover can be placed over the filter in the collection device to prevent air from escaping out of the collection device, and the drain tube can be placed into the reservoir at step 604. The drain valve is opened at step 605 and a pulse is generated at step 606. In an optional embodiment, after the pulse is generated at step 606, if the collection device is not yet empty at step 607, the flow (as shown at point D) can return to step 606. However, if the collection device is empty at step 607, the process proceeds to close the drain valve, open the filter cover, and return to step 502 to wait for the set delay to expire. In another optional embodiment, after the pulse is generated at step 606, if a decrease in pressure is not sensed at step 609 by pressure transducer opposite the gas or air source, the collection device is not yet empty, so the flow (as shown at point E) returns to step 606. However, a pressure decrease is sensed at step 609, then the collection device is empty and the process can proceed to close the drain valve, open the filter cover, and return to step 502 to wait for the set delay to expire.

In each of the herein disclosed embodiments, it is contemplated that features (or process stages) from one embodiment can be used in combination with or can substitute features (or process stages) on another of the disclosed embodiments. Vacuum can also be utilized, e.g., by coupling a vacuum source to the collection device, to assist in removing fluid from the conduit, as is taught in one or more of the prior art documents expressly incorporated by reference herein. In one or more embodiments, the gas can be in the form of a pressure pulse and/or can be continuous gas flow and/or for a predetermined period of time and/or a combination of these. Furthermore, the gas described herein can, in embodiments, be air drawn from the atmosphere immediately surrounding the gas pressure device. Alternatively, the gas can be a gas such as, e.g., nitrogen or oxygen. Other gas can also be utilized provided they function as intended herein.

This invention has been described and specific examples of the invention have been portrayed. While the invention has been described in terms of particular variations and illustrative figures, those of ordinary skill in the art will recognize that the invention is not limited to the variations of figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

What is claimed:

1. A drainage or collection system for biological fluids, comprising:
at least one conduit for transporting a biological fluid from a catheter to a collection device; and
an automated device programmable to automatically supply at least one gas pulse through the at least one conduit and into the collection device.

2. The drainage or collection system in accordance with claim 1, wherein the automated device comprises a programmable microprocessor coupled to control a gas source.

3. The drainage or collection system in accordance with claim 2, wherein the gas source comprises a vacuum pump.

4. The drainage or collection system in accordance with claim 2, wherein the automated device further comprises a pressure transducer structured and arranged to monitor the gas pressure of the at least one gas pulse.

5. The drainage or collection system in accordance with claim 1, wherein the automated device comprises a user interface to program at least one of gas pressure magnitude, gas pulse duration, and period between pulses.

6. The drainage or collection system in accordance with claim 1, further comprising a valve located between the catheter and the container to prevent the at least one gas pulse from flowing toward the catheter.

7. The drainage or collection system in accordance with claim 1, wherein the automated device includes a gas pulse control or regulation device comprising a pressure transducer and a microprocessor.

8. The drainage or collection system in accordance with claim 1, further comprising a transducer positionable at least partially beneath the collection device.

9. The drainage or collection system in accordance with claim 8, wherein an output of the transducer is input to the automated device.

10. The drainage or collection system in accordance with claim 1, wherein the collection device comprises a filter and a closable filter cover.

11. The drainage or collection system in accordance with claim 10, wherein the collection device further comprises a drain tube extending from a bottom of the collection device, the drain tube having an end insertable into a fluid reservoir.

12. The drainage or collection system in accordance with claim 11, wherein the collection device further comprises a high level sensor coupled to the automated device.

13. The drainage or collection system in accordance with claim 11, wherein the collection device further comprises a low level sensor coupled to the automated device.

14. The drainage or collection system in accordance with claim 1, wherein the automated device comprises a signal conditioning circuit structured to receive at least one of bladder pressure and bladder temperature as an input.

15. The drainage or collection system in accordance with claim 14, wherein the signal conditioning circuit is coupled to a gas source structured and arranged to generate the at least one gas pulse.

16. A method for draining or collecting biological fluids, comprising:
guiding biological fluid through at least one conduit from a catheter to a collection device; and
automatically supplying at least one gas pulse through the at least one conduit and into the collection device.

17. The method in accordance with claim 16, wherein the at least one gas pulse forces biological fluids pooling in the at least one conduit into the collection device.

18. The method in accordance with claim 16, wherein the at least one gas pulse forces biological fluids in the collector device out of the collection device.

19. The method in accordance with claim 16, further comprising programming a microprocessor to control a gas source to generate the at least one gas pulse.

20. The method in accordance with claim 19, further comprising inputting at least one of bladder pressure and bladder temperature into a signal conditioning circuit coupled to the gas source.

21. The method in accordance with claim 16, further comprising controlling or regulating a pressure magnitude of the at least one gas pulse.

22. The method in accordance with claim 16, further comprising monitoring a high level sensor of the collection device, and issuing an alert when the biological fluids reach the high level sensor.
